(19) United States
(12) Patent Application Publication
(10) Pub. No.: US 2026/0024612 A1
(43) Pub. Date: Jan. 22, 2026

(54) METHOD AND DEVICE FOR PREDICTING DRUG-TARGET INTERACTION, AND STORAGE MEDIUM

(71) Applicant: BOE Technology Group Co., Ltd., Beijing (CN)

(72) Inventors: Sifan WANG, Beijing (CN); Shuobin LIANG, Beijing (CN)

(73) Assignee: BOE Technology Group Co., Ltd., Beijing (CN)

(21) Appl. No.: 19/099,054

(22) PCT Filed: Aug. 15, 2023
(86) PCT No.: PCT/CN2023/113058

§ 371 Date: Jan. 28, 2025

(30) Foreign Application Priority Data
Aug. 18, 2022 (CN) 202210992939.5

(51) Int. Cl. G16B 15/30; G16B 5/20; G16H 70/40

(52) U.S. Cl. CPC G16B 15/30; G16B 5/20; G16H 70/40

(57) ABSTRACT

A method for predicting drug-target interaction includes: determining a first drug association matrix according to drug attribute information, the drug attribute information including at least one of a drug structure similarity, a pharmacophore similarity, a side effect similarity, and a GO pathway-based similarity of drugs, and the first drug association matrix being used to characterize feature information of each drug on at least one drug attribute; determining a first target association matrix according to target attribute information, the target attribute information including at least one of a target structure similarity and a target interaction relationship of targets, and the first target association matrix being used to characterize feature information of each target on at least one target attribute; and predicting a probability of interaction between a drug and a target according to the first drug association matrix and the first target association matrix.

### CROSS-REFERENCE TO RELATED APPLICATIONS

This application is a national phase entry under 35 USC 371 of International Patent Application No. PCT/CN2023/113058, filed on Aug. 15, 2023, which claims priority to Chinese Patent Application No. 202210992939.5, filed on Aug. 18, 2022, each are incorporated herein by reference in their entirety.

### TECHNICAL FIELD

The present disclosure relates to the field of biomedicine, and in particular to a method for predicting drug-target interaction, a device for predicting drug-target interaction, and a storage medium.

### BACKGROUND

In a process of new drug development, the key step is to determine interaction between a drug and a target protein. Due to the wide variety of drug factors and target proteins, it is inefficient to determine interaction between a drug and a target protein through experiments, which makes it difficult to meet the needs of drug development.

At present, a computer is used to predict the interaction between the drug and target protein in the related art. However, this method cannot accurately extract feature information of the drug and target protein, resulting in low accuracy in predicting the interaction between the drug and target protein.

### SUMMARY

In an aspect, a method for predicting drug-target interaction is provided. The method includes: determining a first drug association matrix according to drug attribute information, the drug attribute information including at least one of a drug structure similarity, a pharmacophore similarity, a side effect similarity, and a gene ontology (GO) pathway-based similarity of a plurality of drugs, and the first drug association matrix being used to characterize feature information of each drug on at least one drug attribute; determining a first target association matrix according to target attribute information, the target attribute information including at least one of a target structure similarity and a target interaction relationship of a plurality of targets, and the first target association matrix being used to characterize feature information of each target on at least one target attribute; and predicting a probability of interaction between a drug and a target according to the first drug association matrix and the first target association matrix.

In some embodiments, determining the first drug association matrix according to the drug attribute information, includes: inputting the drug attribute information and a drug identification vector into a first graph convolution model to obtain an initial drug association matrix, the initial drug association matrix being used to characterize feature information of the plurality of drugs on each drug attribute; and inputting the initial drug association matrix into a second graph convolution model to obtain the first drug association matrix, the second graph convolution model being used to adjust feature information of a drug according to degrees of influence of drug attributes on the drug.

In some embodiments, the method further includes: obtaining a drug attribute vector, the drug attribute vector including at least one of drug structure vectors, pharmacophore vectors, side effect vectors and targeted gene vectors of the plurality of drugs; and determining the drug attribute information according to the drug attribute vector.

In some embodiments, the method further includes: obtaining a drug structure vector of a first drug and a drug structure vector of a second drug, the first drug and the second drug being drugs in the plurality of drugs; and determining a drug structure similarity between the first drug and the second drug according to the drug structure vector of the first drug and the drug structure vector of the second drug.

In some embodiments, the method further includes: obtaining a pharmacophore vector of a first drug and a pharmacophore vector of a second drug, the first drug and the second drug being drugs in the plurality of drugs; and determining a pharmacophore similarity between the first drug and the second drug according to the pharmacophore vector of the first drug and the pharmacophore vector of the second drug.

In some embodiments, the method further includes: obtaining a side effect vector of a first drug and a side effect vector of a second drug, the first drug and the second drug being drugs in the plurality of drugs; and determining a side effect similarity between the first drug and the second drug according to the side effect vector of the first drug and the side effect vector of the second drug.

In some embodiments, the method further includes: obtaining first action targets of a first drug and second action targets of a second drug, the first drug and the second drug being drugs in the plurality of drugs; calculating sequence similarities between the first action targets and the second action targets; matching a first action target and a second action target according to the sequence similarities between the first action targets and the second action targets to obtain at least one action target pair; and determining a GO pathway-based similarity between the first drug and the second drug according to a sequence similarity of the at least one action target pair.

In some embodiments, determining the GO pathway-based similarity between the first drug and the second drug according to the sequence similarity of the at least one action target pair, includes: determine a ratio of a number of action targets in the at least one action target pair to a total number of action targets of the first action targets and the second action targets; and determining the GO pathway-based similarity between the first drug and the second drug according to the sequence similarity of the at least one action target pair and the ratio.

In some embodiments, determining the first target association matrix according to the target attribute information, includes: inputting the target attribute information and a target identification vector into a third graph convolution model to obtain an initial target association matrix, the initial target association matrix being used to characterize feature information of the plurality of targets on each target attribute; and inputting the initial target association matrix into a fourth graph convolution model to obtain the first target association matrix, the fourth graph convolution model being used to adjust feature information of each target according to degrees of influence of target attributes on each target.

In some embodiments, the method further includes: obtaining a target sequence of a first target and a target sequence of a second target, the first target and the second target being targets in the plurality of targets; and determining a target structure similarity between the first target and the second target according to the target sequence of the first target and the target sequence of the second target.

In some embodiments, predicting the probability of interaction between the drug and the target according to the first drug association matrix and the first target association matrix, includes: inputting the first drug association matrix and the first target association matrix into a first fusion model to obtain the probability of interaction between the drug and the target.

In some embodiments, predicting the probability of interaction between the drug and the target according to the first drug association matrix and the first target association matrix, includes: determining a second drug association matrix according to a maximum feature value of each drug on at least one drug attribute in the first drug association matrix, the first drug association matrix including feature values of each drug on the at least one drug attribute; determining a second target association matrix according to a maximum feature value of each target on at least one target attribute in the first target association matrix, the first target association matrix including feature values of each target on the at least one target attribute; and inputting the second drug association matrix and the second target association matrix into a second fusion model to obtain the probability of interaction between the drug and the target.

In another aspect, a non-transitory computer-readable storage medium is provided. The computer-readable storage medium has stored computer program instructions that, when run on a computer, cause the computer to perform: determining a first drug association matrix according to drug attribute information, the drug attribute information including at least one of a drug structure similarity, a pharmacophore similarity, a side effect similarity, and a gene ontology (GO) pathway-based similarity of a plurality of drugs, and the first drug association matrix being used to characterize feature information of each drug on at least one drug attribute; determining a first target association matrix according to target attribute information, the target attribute information including at least one of a target structure similarity and a target interaction relationship of a plurality of targets, and the first target association matrix being used to characterize feature information of each target on at least one target attribute; and predicting a probability of interaction between a drug and a target according to the first drug association matrix and the first target association matrix.

In some embodiments, determining the first drug association matrix according to the drug attribute information, includes: inputting the drug attribute information and a drug identification vector into a first graph convolution model to obtain an initial drug association matrix, the initial drug association matrix being used to characterize feature information of the plurality of drugs on each drug attribute; and inputting the initial drug association matrix into a second graph convolution model to obtain the first drug association matrix, the second graph convolution model being used to adjust feature information of a drug according to degrees of influence of drug attributes on the drug.

In some embodiments, the instructions cause the computer to further perform: obtaining a drug attribute vector, the drug attribute vector including at least one of drug structure vectors, pharmacophore vectors, side effect vectors and targeted gene vectors of the plurality of drugs; and determining the drug attribute information according to the drug attribute vector.

In some embodiments, determining the first target association matrix according to the target attribute information, includes: inputting the target attribute information and a target identification vector into a third graph convolution model to obtain an initial target association matrix, the initial target association matrix being used to characterize feature information of the plurality of targets on each target attribute; and inputting the initial target association matrix into a fourth graph convolution model to obtain the first target association matrix, the fourth graph convolution model being used to adjust feature information of each target according to degrees of influence of target attributes on each target.

In some embodiments, predicting the probability of interaction between the drug and the target according to the first drug association matrix and the first target association matrix, includes: inputting the first drug association matrix and the first target association matrix into a first fusion model to obtain the probability of interaction between the drug and the target.

In some embodiments, predicting the probability of interaction between the drug and the target according to the first drug association matrix and the first target association matrix, includes: determining a second drug association matrix according to a maximum feature value of each drug on at least one drug attribute in the first drug association matrix, the first drug association matrix including feature values of each drug on the at least one drug attribute; determining a second target association matrix according to a maximum feature value of each target on at least one target attribute in the first target association matrix, the first target association matrix including feature values of each target on the at least one target attribute; and inputting the second drug association matrix and the second target association matrix into a second fusion model to obtain the probability of interaction between the drug and the target.

In yet another aspect, a device for predicting drug-target interaction is provided. The device includes a processor and a memory; the memory is used to store computer programs or instructions, and the processor is used to run the computer programs or instructions to implement the method for predicting drug-target interaction as described in any of the above embodiments.

It should be noted that, the computer instructions may be stored in whole or in part on a computer-readable storage medium. The computer-readable storage medium may be packaged together with the processor of the device, or may be packaged separately from the processor of the device, which is not limited in the disclosure.

In the present disclosure, the name of the device for predicting drug-target interaction does not limit devices or functional modules themselves. In actual implementation, these devices or functional modules may appear under other names. As long as the function of each device or functional module is similar to that of the present disclosure, it falls within the scope of the claims of the present disclosure and its equivalent technology.

### BRIEF DESCRIPTION OF THE DRAWINGS

In order to describe technical solutions in the present disclosure more clearly, the accompanying drawings to be used in some embodiments of the present disclosure will be introduced briefly. Obviously, the accompanying drawings to be described below are merely accompanying drawings of some embodiments of the present disclosure, and a person of ordinary skill in the art can obtain other drawings according to these drawings. In addition, the accompanying drawings to be described below may be regarded as schematic diagrams, and are not limitations on actual sizes of products, actual processes of methods and actual timings of signals involved in the embodiments of the present disclosure.

FIG. 1 is a structural diagram of a system for predicting drug-target interaction, in accordance with some embodiments;

FIG. 2 is a flowchart of a method for predicting drug-target interaction, in accordance with some embodiments;

FIG. 3 is a structural diagram of a first drug association matrix, in accordance with some embodiments;

FIG. 4 is a flowchart of a pooling operation, in accordance with some embodiments;

FIG. 5 is a flowchart of a data fusion method, in accordance with some embodiments;

FIG. 6 is a flowchart of another method for predicting drug-target interaction, in accordance with some embodiments;

FIG. 7 is a flowchart of a method for determining a first drug association matrix, in accordance with some embodiments;

FIG. 8 is a structural diagram of drug attribute information, in accordance with some embodiments;

FIG. 9 is a flowchart of yet another method for predicting drug-target interaction, in accordance with some embodiments;

FIG. 10 is a flowchart of yet another method for predicting drug-target interaction, in accordance with some embodiments;

FIG. 11 is a flowchart of yet another method for predicting drug-target interaction, in accordance with some embodiments;

FIG. 12 is a flowchart of yet another method for predicting drug-target interaction, in accordance with some embodiments;

FIG. 13 is a structural diagram of a device for predicting drug-target interaction, in accordance with some embodiments; and

FIG. 14 is a structural diagram of another device for predicting drug-target interaction, in accordance with some embodiments.

### DETAILED DESCRIPTION

Technical solutions in some embodiments of the present disclosure will be described clearly and completely below with reference to the accompanying drawings. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as open and inclusive meaning, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials or characteristics may be included in any one or more embodiments or examples in any suitable manner.

Hereinafter, the terms “first” and “second” are used for descriptive purposes only, and are not to be construed as indicating or implying the relative importance or implicitly indicating the number of indicated technical features. Thus, a feature defined with “first” or “second” may explicitly or implicitly include one or more of the features. In the description of the embodiments of the present disclosure, the term “a/the plurality of” means two or more unless otherwise specified.

In the description of some embodiments, the terms such as “coupled” and “connected” and derivatives thereof may be used. For example, the term “connected” may be used in the description of some embodiments to indicate that two or more components are in direct physical or electrical contact with each other. As another example, the term “coupled” may be used in the description of some embodiments to indicate that two or more components are in direct physical or electrical contact. However, the term “coupled” or “communicatively coupled” may also mean that two or more components are not in direct contact with each other, but still cooperate or interact with each other. The embodiments disclosed herein are not necessarily limited to the content herein.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

As used herein, the term “if”, depending on the context, is optionally construed as “when”, “in a case where”, “in response to determining”, or “in response to detecting”. Similarly, depending on the context, the phrase “if it is determined” or “if [a stated condition or event] is detected” is optionally construed as “in a case where it is determined”, “in response to determining”, “in a case where [the stated condition or event] is detected”, or “in response to detecting [the stated condition or event]”.

The phrase “applicable to” or “configured to” as used herein indicates an open and inclusive expression, which does not exclude devices that are applicable to or configured to perform additional tasks or steps.

In addition, the phrase “based on” as used herein is meant to be open and inclusive, since a process, step, calculation or other action that is “based on” one or more of the stated conditions or values may, in practice, be based on additional conditions or values beyond those stated.

As used herein, the term such as “about”, “substantially” or “approximately” includes a stated value and an average value within an acceptable range of deviation of a particular value. The acceptable range of deviation is determined by a person of ordinary skill in the art in view of the measurement in question and errors associated with the measurement of a particular quantity (i.e., the limitation of the measurement system).

In the following, terms involved in the embodiments of the present disclosure are explained to facilitate readers' understanding.

### (1) Gene Ontology (GO)

The gene ontology includes three parts: molecular function (MF), cellular component (CC) and biological process (BP).

The molecular function refers to the activity at the molecular level of a single gene product (such as protein or ribonucleic acid (RNA)) or a complex of multiple gene products. The cellular component refers to a cellular structural location where the gene product performs its function. The biological process refers to a biological process accomplished through a variety of molecular activities.

Basic elements in the gene ontology include: identification information (ID), aspect (such as the molecular function, cellular component or biological process), definition information, and relationship.

### (2) Neural Network

Neural networks (NNs), also known as artificial neural networks (ANNs), are mathematical model algorithms that mimic behavioral characteristics of animal neural networks and perform distributed parallel information processing. The neural networks include deep learning networks, such as convolutional neural networks (CNN), residual network (ResNet), long short-term memory network (LSTM).

In a process of new drug development, the key step is to determine interaction between a drug and a target protein. Due to the wide variety of drug factors and target proteins, it is inefficient to determine interaction between a drug and a target protein through experiments, which makes it difficult to meet the needs of drug development.

At present, a computer is used to predict the interaction between the drug and target protein in the related art. However, this method cannot accurately extract feature information of the drug and target protein, resulting in low accuracy in predicting the interaction between the drug and target protein.

In light of this, embodiments of the present disclosure provide a method for predicting interaction between a drug and a target, in which a first drug association matrix is determined according to drug attribute information, a first target association matrix is determined according to target attribute information, and then a probability of interaction between the drug and the target is predicted based on the first drug association matrix and the first target association matrix. Therefore, the embodiments of the present disclosure may accurately extract feature information of the drug and feature information of the target based on multiple attribute dimensions, thereby improving the accuracy of predicting the interaction between the drug and the target.

Implementations in embodiments of the present disclosure will be described in details below with reference to the accompanying drawings of the specification.

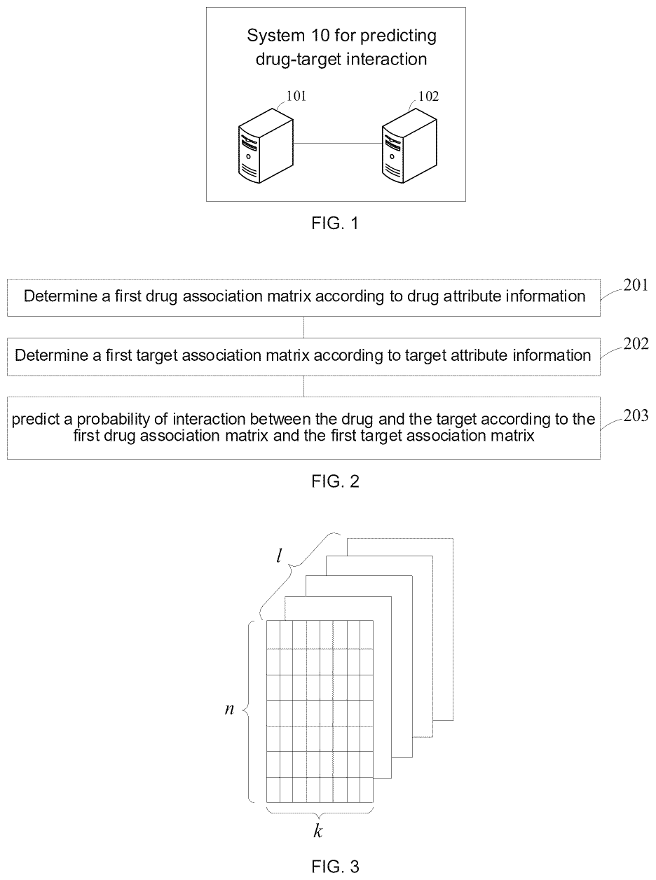

FIG. 1 is a structural diagram of a system 10 for predicting drug-target interaction, in accordance with some embodiments. As shown in FIG. 1, the system 10 for predicting the drug-target interaction includes a device 101 for predicting the drug-target interaction (referred to as a prediction device 101 below) and a data server 102.

The prediction device 101 and the data server 102 are connected through a communication link, which may be a wired communication link or a wireless communication link. The present disclosure is not limited thereto.

It should be noted that the prediction device 101 and the data server 102 may be separate electronic devices. For example, the prediction device 101 and the data server 102 are servers. The prediction device 101 and the data server 102 may also be application programs for realizing the function of predicting a probability of interaction between a drug and a target.

Alternatively, the prediction device 101 and the data server 102 may also be processing chips or functional modules in a same device. In this case, the information interaction between the prediction device 101 and the data server 102 is an internal interaction in the same device.

For example, the data server 102 includes a processor, which may be a general-purpose central processing unit (CPU), a microprocessor, an application specific integrated circuit (ASIC), or one or more integrated circuits for controlling execution of programs of solutions in the present disclosure.

The data server 102 further includes a transceiver, which may be a device that use any type of transceiver, and is used to communicate with other devices or communication networks, such as Ethernet, radio access network (RAN), and wireless local area network (WLAN).

The data server 102 further includes a memory, which may be, but is not limited to, a read-only memory (ROM) or a static storage device of any other type that can store static information and instructions, a random access memory (RAM) or a dynamic storage device of any other type that can store information and instructions, an electrically erasable programmable read-only memory (EEPROM), a compact disc read-only memory (CD-ROM), or any other compact disc storage or optical disc storage (including a compact disc, a laser disc, an optical disc, a digital general-purpose disc, or a Blue-ray disc), a magnetic disk storage medium or any other magnetic storage device, or any other medium that can be used to carry or store desired program codes in a form of instructions or data structures and that can be accessed by a computer. The memory may exist independently and be connected to the processor through a communication line. The memory may also be integrated with the processor.

The prediction device 101 is configured to determine the first drug association matrix according to the drug attribute information.

The drug attribute information includes at least one of a drug structure similarity, a pharmacophore similarity, a side effect similarity, and a GO pathway-based similarity of a plurality of drugs. The first drug association matrix is used to characterize feature information of each drug on at least one drug attribute.

It should be noted that, the drug structure similarity refers to a degree of similarity between the drugs in chemical structure, the pharmacophore similarity refers to a degree of similarity between the drugs in pharmacophore composition, the side effect similarity refers to a degree of similarity between side effects that exist in the drugs, and the GO pathway-based similarity refers to a degree of similarity between targets that the drugs can act on.

The prediction device 101 is further configured to determine the first target association matrix according to the target attribute information.

The target attribute information includes at least one of a target structure similarity of a plurality of targets and a target interaction relationship of the targets. The first target association matrix is used to characterize feature information of each target on at least one target attribute.

It should be noted that, the target structure similarity refers to a degree of similarity between the targets in chemical structure, and the target interaction relationship refers to whether there is interaction between the targets.

For example, the chemical structure of the target includes at least one of a primary structure, secondary structure, tertiary structure and quaternary structure. The primary structure is an amino acid sequence of the target; the secondary structure is a regular fragment formed by folding of the protein; the tertiary structure is a specific spatial structure generated by coiling and folding of the protein on the basis of the secondary structure; and the quaternary structure refers to a spatial structure formed by the interaction of a plurality of peptide chains.

The prediction device 101 is further configured to predict the probability of interaction between the drug and the target according to the first drug association matrix and the first target association matrix.

The prediction device 101 is further configured to: obtain drug data and target data from the data server 102, determine the drug attribute information according to the drug data, and determine the target attribute information according to the target data.

The drug data includes the drug structure, pharmacophore composition and existing side effect(s) of each drug, and target(s) that each drug can act on. The target data includes the target structure of each target and the target interaction relationship.

The data server 102 is configured to store the drug data and target data, and send the drug data and target data to the prediction device 101.

It should be noted that, the embodiments of the present disclosure may refer to each other. For example, the same or similar steps, method embodiments, system embodiments and device embodiments can refer to each other, which will not be limited.

FIG. 2 is a flowchart of a method for predicting interaction between a drug and a target, in accordance with some embodiments. As shown in FIG. 2, the method includes the following steps 201 to 203.

In step 201, the prediction device determines a first drug association matrix according to drug attribute information.

The drug attribute information includes at least one of a drug structure similarity, a pharmacophore similarity, a side effect similarity, and a GO pathway-based similarity of a plurality of drugs. The first drug association matrix is used to characterize feature information of each drug on at least one drug attribute.

It should be noted that, the drug structure similarity refers to the degree of similarity between the drugs in chemical structure, the pharmacophore similarity refers to the degree of similarity between the drugs in pharmacophore composition, the side effect similarity refers to the degree of similarity between side effects that exist in the drugs, and the GO pathway-based similarity refers to the degree of similarity between targets that the drugs can act on.

For example, the drug attribute information is expressed in the form of a table or matrix, for example, as shown in Table 1 below.

TABLE 1
Drug structure similarity table

| | Drug 1 | Drug 2 | . . . | Drug n |
|---|---|---|---|---|
| Drug 1 | 1 | 0.2 | . . . | 0.12 |
| Drug 2 | 0.2 | 1 | . . . | 0.01 |
| . . . | . . . | . . . | . . . | . . . |
| Drug n | 0.12 | 0.01 | . . . | 1 |

Table 1 is used to represent the drug structure similarity between n drugs. The drug structure similarity between Drug 1 and Drug 1 is 1, the drug structure similarity between Drug 1 and Drug 2 is 0.2, and so on. For the pharmacophore similarity, side effect similarity, and GO pathway-based similarity, reference may be made to the expression of the drug structure similarity, which will not be repeated here. n is a positive integer.

For example, in the first drug association matrix, the feature information of the drug on at least one drug attribute may be represented by feature values, such as specific numerical values, vectors, or higher-dimensional representation. Alternatively, the feature information may be represented by text data, such as a string. The present disclosure is not limited thereto.

In an example where the feature information is a vector, as shown in FIG. 3, the first drug association matrix is an n*k*l third-order tensor, which includes feature information of n drugs on/drug attributes. This feature information is the k-dimensional vector.

In a possible implementation, the prediction device may input the drug attribute information into a neural network model based on a neural network algorithm to obtain the first drug association matrix.

For example, the neural network algorithm may be a convolutional neural network (e.g., a graph convolutional neural network) or a long short-term memory (LSTM) neural network.

In step 202, the prediction device determines a first target association matrix according to target attribute information.

The target attribute information includes at least one of a target structure similarity of a plurality of targets and a target interaction relationship of the targets. The first target association matrix is used to characterize feature information of each target on at least one target attribute. The target interaction relationship of the targets can be obtained from the String database by the prediction device.

It should be noted that, the target structure similarity refers to the degree of similarity between the targets in chemical structure, and the target interaction relationship refers to whether there is interaction between the targets.

For example, the chemical structure of the target includes at least one of the primary structure, secondary structure, tertiary structure and quaternary structure. The primary structure is an amino acid sequence of the target; the secondary structure is a regular fragment formed by folding of the protein; the tertiary structure is a specific spatial structure generated by coiling and folding of the protein on the basis of the secondary structure; and the quaternary structure refers to a spatial structure formed by the interaction of a plurality of peptide chains.

The target attribute information can be expressed in the form of a table or matrix. For the target structure similarity, reference may be made to the drug structure similarity in the above step 201, which will not be repeated here. The target interaction relationship is shown in Table 2 below.

TABLE 2
Target interaction relationship table

| | Target 1 | Target 2 | . . . | Target m |
|---|---|---|---|---|
| Target 1 | 0 | 1 | . . . | 0 |
| Target 2 | 1 | 0 | . . . | 1 |
| . . . | . . . | . . . | . . . | . . . |
| Target m | 0 | 1 | . . . | 0 |

Table 2 is used to represent the interaction relationship between m targets. There is no interaction relationship between Target 1 and Target 1, which is represented by “0”; there is an interaction relationship between Target 1 and Target 2, which is represented by “1”, and so on. m is a positive integer.

For the first target association matrix, reference may be made to the first drug association matrix in the above step 201, which will not be repeated here.

In a possible implementation, the prediction device may input the target attribute information into a neural network model based on a neural network algorithm to obtain the first target association matrix.

For example, the neural network algorithm may be a convolutional neural network (e.g., a graph convolutional neural network) or a long short-term memory (LSTM) neural network.

It should be noted that the steps 201 and 202 are independent of each other. The step 201 may be executed before the step 202, may be executed after the step 202, or may be executed in parallel with the step 202. FIG. 2 only illustrates an example in which the step 201 is executed before the step 202, for describing the method for predicting the interaction between the drug and the target provided in the embodiments of the present disclosure. The execution sequence of the steps is not limited in the present disclosure.

In step 203, the prediction device predicts a probability of interaction between the drug and the target according to the first drug association matrix and the first target association matrix.

In a possible implementation, the prediction device inputs the first drug association matrix and the first target association matrix into a first fusion model to obtain the probability of the interaction between the drug and the target.

The first fusion model may be a neural network model. The first fusion model is used to calculate the probability of the interaction between the drug and the target according to a first weight. The first weight can be obtained by training sample data.

In another possible implementation, the prediction device performs a pooling operation on the first drug association matrix and the first target association matrix to obtain a second drug association matrix and a second target association matrix, and predicts the probability of the interaction between the drug and the target according to the second drug association matrix and the second target association matrix.

The pooling operation may be average pooling, max pooling, global average pooling, or global max pooling.

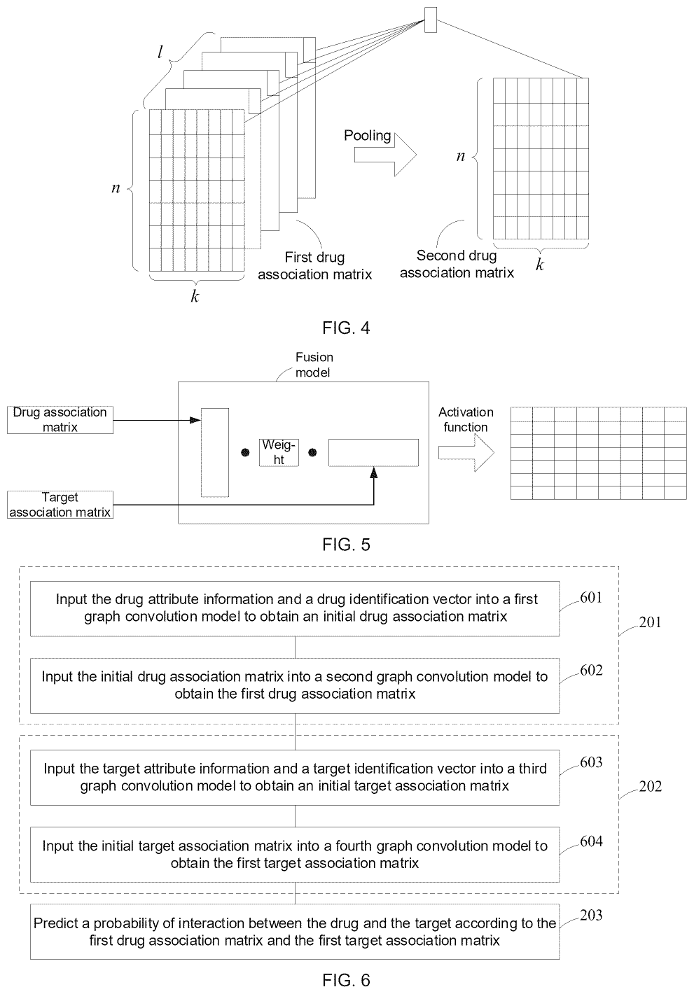

As shown in FIG. 4, the first drug association matrix is an n*k*l third-order tensor, which includes feature information of n drugs on/drug attributes. This feature information is the k-dimensional vector. The prediction device determines a feature value of the i-th drug on the j-th dimension in the second drug association matrix according to/feature values of the i-th drug on the j-th dimension. For example, the max pooling can use the maximum value in the/feature values of the i-th drug on the j-th dimension as the feature value of the i-th drug on the j-th dimension in the second drug association matrix. The average pooling can use the average value of the/feature values of the i-th drug on the j-th dimension as the feature value of the i-th drug on the j-th dimension in the second drug association matrix.

It should be noted that, the prediction device may reduce the dimensions of the first drug association matrix and the first target association matrix through the pooling operation. In addition, the second drug association matrix obtained after pooling can characterize the comprehensive feature information of each drug, and the second target association matrix obtained after pooling can characterize the comprehensive feature information of each target. Therefore, based on the pooling operation, the prediction device may accurately extract the feature information of the drug and the target, and reduce the complexity of predicting the probability of the interaction between the drug and the target, improving the prediction efficiency.

The max pooling is taken as an example, as a possible implementation, the prediction device determines the second drug association matrix according to the maximum feature value of each drug on at least one drug attribute in the first drug association matrix, and determines the second target association matrix according to the maximum feature value of each target on at least one target attribute in the first target association matrix. The prediction device inputs the second drug association matrix and the second target association matrix into a second fusion model to obtain the probability of the interaction between the drug and the target.

The first drug association matrix includes feature values of each drug on at least one drug attribute, and the first target association matrix includes feature values of each target on at least one target attribute. The second fusion model may be a neural network model. The second fusion model is used to calculate the probability of the interaction between the drug and the target according to a second weight. The second weight can be obtained by training sample data.

Based on the above technical solutions, in the method for predicting the interaction between the drug and the target provided in the embodiments of the present disclosure, the prediction device determines the first drug association matrix according to the drug attribute information, determines the first target association matrix according to the target attribute information, and then predicts the probability of the interaction between the drug and the target based on the first drug association matrix and the first target association matrix. Since the drug attribute(s) in the embodiments of the present disclosure include at least one of the drug structure similarity, pharmacophore similarity, side effect similarity, and GO pathway-based similarity, and the target attribute information includes at least one of the target structure similarity of the targets and the target interaction relationship of the targets, it is determined that the first drug association matrix can characterize the feature information of each drug on at least one drug attribute, and the first target association matrix can characterize the feature information of each target on at least one target attribute. In this way, the embodiments of the present disclosure may more accurately extract the feature information of the drug and the target, thereby improving the accuracy of predicting the interaction between the drug and the target.

The following uses the neural network model as an example to illustrate the process of the prediction device predicting the probability of the interaction between the drug and the target.

As shown in FIG. 5, the prediction device inputs the drug association matrix and the target association matrix into the fusion model, and obtains the probability of the interaction between the drug and the target through weight calculation and activation function mapping.

The drug association matrix may be the first drug association matrix or the second drug association matrix described above, and the target association matrix may be the first target association matrix or the second target association matrix described above. The fusion model may be the first fusion model or the second fusion model described above. The weight coefficient can be determined through model training based on sample data, which will not be described in detail in the present disclosure.

For example, the drug association matrix Y_{d} is a second-order tensor with n*k dimensions, i.e., Y_{d}∈R^{n·k}. The target association matrix Yₚ is a second-order tensor with m*k dimensions, i.e., Yₚ∈R^{m·k}. k is a positive integer.

The probability of the interaction between the drug and the target satisfies the following formula 1.

$$P = σ ( {Y}_{d} {W}^{′} {Y}_{p}^{T} ) \qquad Formula 1$$

P is the probabilities of interaction between n drugs and m targets, and P∈R^{n·m}. In σ( ) is the activation function (e.g., sigmoid function). Y_{d} is the drug association matrix, and Yₚ^{T} is the transpose matrix of the target association matrix. W′ is the weight coefficient of the fusion model, W′∈R^{k·k}.

The process of determining the first drug association matrix by the prediction device will be described below in combination with the above step 201.

As a possible embodiment of the present disclosure, as shown in FIGS. 2 and 6, the step 201 can be implemented through the following steps 601 to 602.

In step 601, the prediction device inputs the drug attribute information and a drug identification vector into a first graph convolution model to obtain an initial drug association matrix.

The initial drug association matrix is used to characterize feature information of the plurality of drugs on each drug attribute. The drug identification vector is used to identify each of the plurality of drugs.

For example, the drug identification vector includes a drug structure vector of each of the plurality of drugs. The drug structure vector is used to characterize the drug structure of a corresponding drug. The drug structure is the binary chemical structure of the drug, e.g., corresponding to an 881-dimensional vector that meets the simplified molecular input line entry system (SMILES) standard.

As shown in Table 3 below, it is a corresponding relationship table between vector positions in the drug structure vector and types of structures.

TABLE 3
Corresponding relationship table between drug structure
vector positions and types of structures

| Drug structure | |
|---|---|
| vector position | Structure type |
| 0 | >=4H |
| 1 | >=8H |
| . . . | . . . |
| 284 | C—C |
| . . . | . . . |
| 425 | P═O |
| . . . | . . . |
| 880 | BrC1C(Br)CCC1 |

The binary chemical structure of the drug includes 881 types of structures, which respectively correspond to 881 vector positions in the drug structure vector. When a certain type of structures exists in the drug structure of the drug, the number of the type of structures is on the vector position corresponding to the type of structures. For example, when the drug includes three structures with the type of “>=4H”, the value at the vector position “0” in the drug structure vector corresponding to the drug is “3”.

Alternatively, “1” is used to indicate that the type of structures corresponding to the vector position exists in the drug structure of the drug, and “0” is used to indicate that the type of structures corresponding to the vector position does not exist in the drug structure of the drug. For example, when the structure with the type of “>=4H” exists in the drug, the value at the vector position “0” in the drug structure vector corresponding to the drug is “1”. For example, when the structure with the type of “>=8H” does not exist in the drug, the value at the vector position “1” in the drug structure vector corresponding to the drug is “0”.

The drug acamprosate is taken as an example, the chemical structural formula of the drug according to the SMILES standard is CC(=O)NCCCS(=O)(=O)O, and then the drug structure vector corresponding to the drug is 110000000110001 0 . . . 00000000.

The first graph convolution model is used to determine the feature information of the drugs on each drug attribute according to the drug identification vector and the drug attribute information.

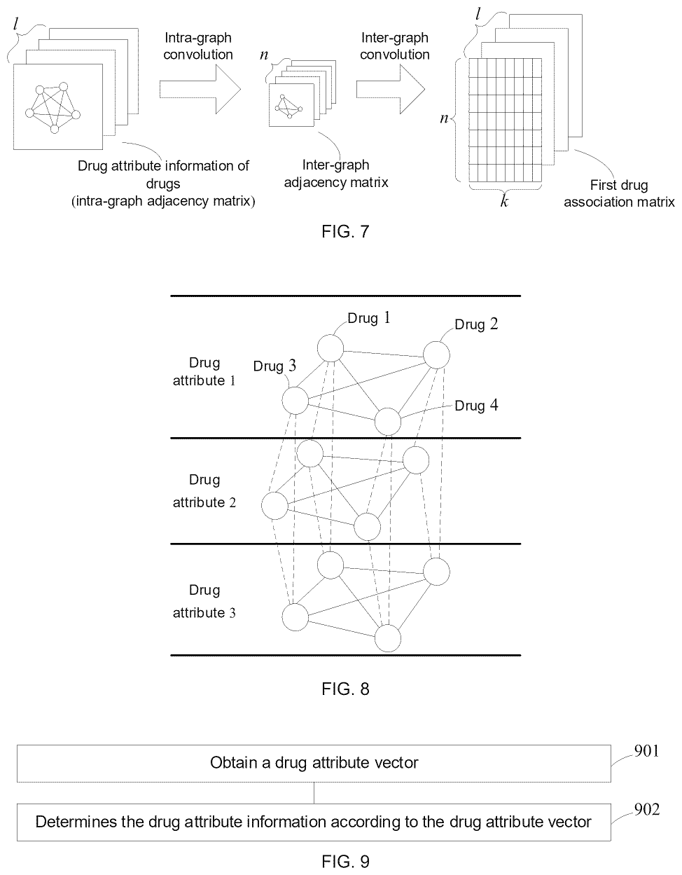

The step 601 is also called an intra-graph convolution operation. As shown in FIG. 7, the prediction device inputs/drug attributes in the drug attribute information (i.e., intra-graph adjacency matrix) of drugs and the drug identification vector (not shown in FIG. 7) as input data into the first graph convolution model to perform intra-graph convolution, so as to obtain the initial drug association matrix (not shown in FIG. 7).

For example, the initial drug association matrix satisfies the following formula 2.

$${Y}_{0} = {D}^{- 1 / 2} A {D}^{- 1 / 2} {H}_{0} {W}_{0} \qquad Formula 2$$

Y₀ is the initial drug association matrix, A is the drug attribute information (i.e., the intra-graph adjacency matrix), D is the degree matrix of the drug attribute information, H₀ is the drug identification vector, and W₀ is the weight coefficient in the first graph convolution model.

The number fields to which the above parameters belong are respectively Y₀∈R^{l·k·n}, A∈R^{n·n·l}, D∈R^{n·n·l}, H₀∈R^{n·i·l}, W₀∈R^{i·k·l}. l is the number of drug attributes in the drug attribute information, n is the number of drugs, k is the dimension parameter, and i is the length of the drug structure vector in the drug identification vector.

For example, the drug attribute information includes drug structure similarity, pharmacophore similarity, side effect similarity and GO pathway-based similarity of drugs, and thus l is 4; the drug structure vector of the drug is an 881-dimensional vector, and thus i is 881; k can be set according to the actual situation. The weight coefficient W₀ can be determined through model training based on sample data, which will not be described in detail in the present disclosure.

Based on the above process, since the drug attribute information includes the association relationship of drugs on the drug attributes, the prediction device may adjust the drug identification vector according to each drug attribute, thereby determining the feature information of drugs on each drug attribute. That is, the step 601 is used to determine, for each drug attribute, the feature information of drugs on the drug attribute.

As shown in FIG. 8, there are three drug attributes (that is, l is 3), and the number of drugs is 4. The node connection relationship connected by the solid lines in the figure is used to represent the association relationship between drugs on one of the drug attributes. In an example where Drug attribute 1 is the drug structure similarity, each node represents the drug structure similarity between the drug itself and itself, the connection between Drug 1 and Drug 2 represents the drug structure similarity between Drug 1 and Drug 2, and so on.

In this way, the prediction device may calculate feature information of the four drugs on the Drug attribute 1 according to similarities between the four drugs on the Drug attribute 1, calculate feature information of the four drugs on Drug attribute 2 according to similarities between the four drugs on Drug attribute 2, and calculate feature information of the four drugs on Drug attribute 3 according to similarities between the four drugs on Drug attribute 3.

In step 602, the prediction device inputs the initial drug association matrix into a second graph convolution model to obtain the first drug association matrix.

The second graph convolution model is used to adjust the feature information of the drug according to degrees of influence of drug attributes on the drug.

The step 602 is also called an inter-graph convolution operation. As shown in FIG. 7, the prediction device inputs the initial drug association matrix determined in the step 601 into the second graph convolution model to perform the inter-graph convolution. That is, the feature information of the same drug on the drug attributes is connected through the inter-graph adjacency matrix, and the connected feature information is adjusted by the weight coefficient and activation function in the second graph convolution model, thereby obtaining the first drug association matrix.

For example, the first drug association matrix satisfies the following formula 3.

$$= σ ( {\tilde{D}}^{- 1 / 2} \tilde{A} {\tilde{D}}^{- 1 / 2} {Y}_{0} ) \qquad Formula 3$$

[symbol] is the first drug association matrix, Ã is the inter-graph adjacency matrix, \tilde{D} is the degree matrix of the inter-graph adjacency matrix, Y₀ is the initial drug association matrix, [symbol] is the weight coefficient in the second graph convolution model, and σ( ) is the activation function (such as relu activation function).

The number fields to which the above parameters belong are respectively Y₀ E [symbol] ∈R^{n·k·l}, Ã∈R^{l·l·n}, \tilde{D}∈R^{l·l·n}, Y₀∈R^{l·k·n}, [symbol] ∈R^{k·k·n}. l is the number of drug attributes in the drug attribute information, n is the number of drugs, and k is the dimension parameter. The weight coefficient [symbol] can be determined through model training based on sample data, which will not be described in detail in the present disclosure.

It should be noted that, Ã is a matrix with all values equal to 1, and is used to connect the feature information of the same drug on the drug attributes. In this case, there is an association relationship between the drug attributes of the drug. In the embodiments of the present disclosure, “0” can also be used to represent that there is no association relationship between two drug attributes, which may be set according to actual situations, and the embodiments of the present disclosure are not limited thereto.

Based on the above process, the prediction device may connect the feature information of the same drug on the drug attributes through the inter-graph adjacency matrix, and adjust the feature information of the drug on the drug attributes based on the weight coefficient for each drug.

For each drug, the degrees of influence of the drug attributes on the drug are different. For example, for Drug 1, the degree of influence of the side effect is relatively high, while the degree of influence of the pharmacophore is a relatively low. For Drug 2, the degree of influence of the side effect is relatively low, while the degree of influence of the pharmacophore is a relatively high. This results in that the feature information of the drugs determined based on the association relationship between the drugs on the same drug attribute cannot accurately represent actual information of the drugs.

However, in the embodiments of the present disclosure, when determining the first drug association matrix, the prediction device may establish the connection relationship between the feature information of the same drug on different drug attributes, and adjust the feature information of the drug based on the degrees of influence of the drug attributes on the drug, so that the extracted feature information of the drug is more accurate.

As shown in FIG. 8, there are three drug attributes (that is, l is 3), and the number of drugs is 4. The node connection relationship connected by the dotted lines in the figure is used to represent the association relationship of the same drug on different drug attributes. Drug 1 is taken as an example, Drug 1 for Drug attribute 1 is connected to Drug 1 for Drug attribute 2 and Drug 1 for Drug attribute 3, which means that for Drug 1, there is the association relationship between Drug attribute 1, Drug attribute 2 and Drug 3.

In this way, the prediction device may adjust the feature information of the drug on the three drug attributes based on the degrees of influence of the three drug attributes on each drug.

Based on the above technical solution, in the embodiments of the present disclosure, the prediction device may first perform the intra-graph convolution operation through the association relationship of the drugs on the same drug attribute and the drug identification vector to obtain the initial drug association matrix. The initial drug association matrix is used to characterize the feature information of the drugs on each drug attribute. Then, the prediction device may perform the inter-graph convolution operation on the initial drug association matrix (i.e., adjust the feature information of each drug according to the degrees of influence of the drug attributes on each drug), so as to make the extracted feature information of the drug more accurate.

It should be noted that, in the embodiments of the present disclosure, the prediction device may perform the steps 601 and 602 multiple times to extract deep feature information of the drug, for example, intra-graph convolution-inter-graph convolution-intra-graph convolution-inter-graph convolution. The data H₀ input in the second intra-graph convolution is the matrix \tilde{Y}₀ output in the first inter-graph convolution. For the implementation of the second inter-graph convolution and the second intra-graph convolution, reference may be made to the steps 601 and 602, which will not be described in detail in the present disclosure.

The process of determining the first targe association matrix by the prediction device will be described below in combination with the step 202.

As a possible embodiment of the present disclosure, as shown in FIGS. 2 and 6, the step 202 can be implemented through the following steps 603 to 604.

In step 603, the prediction device inputs the target attribute information and a target identification vector into a third graph convolution model to obtain an initial target association matrix.

The initial target association matrix is used to characterize the feature information of the plurality of targets on each target attribute.

The target identification vector is used to identify each target. The target identification vector may include target sequence frequency vectors of the targets.

For example, the target sequence is any one of a primary structure sequence, a secondary structure sequence, a tertiary structure sequence or a quaternary structure sequence.

The primary structure sequence is taken as an example, the target contains 20 types of amino acids, each type of amino acids is represented by a letter. In this field, amino acids are usually divided into 7 categories according to their physical and chemical properties, which are: {A, G, V}, {I, L, F, P}, {Y, M, T, S}, {H, N, Q, W}, {R, K}, {D, E} and {C}. The 7 categories of amino acids may be represented by numbers 1 to 7.

For example, Target sequence ALQDVG is represented by “124611”. In addition, the target sequence may be encoded by the K-mer statistical method, where K refers to the smallest tuple in the target sequence. For example, the 3-mers of the target sequence are: 124, 246, 461, and 611.

In this case, the target sequence frequency vector may be expressed in terms of the frequency of each 3-mer. Types of 3-mers include 7*7*7=343 types.

As shown in Table 4 below.

TABLE 4
Relationship table between the type, number
and frequency of 3-mers of the target

| | | | | | | | |
|---|---|---|---|---|---|---|---|
| Type | 111 | . . . | 135 | . . . | 274 | . . . | 777 |
| Number | 321 | | 835 | | 34 | | 85 |
| Frequency | 0.214 | | 0.556 | | 0.023 | | 0.057 |

The target includes 1500 3-mers, and the target sequence frequency vector consists of the frequency of each type of 3-mers. For the targe, the number of 3-mers with the type of “111” is 321, so that the frequency of the 3-mers with the type of “111” is 321/1500=0.214; and so on.

For relevant content of the initial target association matrix, reference may be made to the description of the initial drug association matrix in the step 601, which will not be described in detail here.

In step 604, the prediction device inputs the initial target association matrix into a fourth graph convolution model to obtain the first target association matrix.

The fourth graph convolution model is used to adjust the feature information of each target according to degrees of influence of the target attributes on each target.

For relevant content, reference may be made to the description in the step 602, which will not be described in detail.

Based on the above technical solution, the prediction device in the embodiments of the present disclosure may first perform the intra-graph convolution operation through the target attribute information of the targets on the same target attribute and target identification vector to obtain the initial target association matrix. The initial target association matrix is used to characterize the feature information of the targets on each target attribute. Then, the prediction device may perform the inter-graph convolution operation on the initial target association matrix (i.e., adjust the feature information of each target according to the degrees of influence of the target attributes on each target), so that the extracted feature information of the target is more accurate.

The process of determining the drug attribute information by the prediction device will be described below.

As a possible embodiment of the present disclosure, as shown in FIG. 9, before the prediction device determines the first drug association matrix, the method further includes the following steps 901 and 902.

In step 901, the prediction device obtains a drug attribute vector.

The drug attribute vector includes at least one of drug structure vectors, pharmacophore vectors, side effect vectors and targeted gene vectors of the drugs.

For the drug structure vector, reference may be made to the relevant description in the step 601, which will not be described in detail here.

The pharmacophore vector is used to characterize fingerprint information of the pharmacophore of the drug. The pharmacophore is a specific structure of drug molecules that have relevant characteristics and interactions required for activity, for a given target.

In a possible implementation, the prediction device can encode structural features of the drug molecules through a sub-structure-based fingerprint manner, and classify the structural features based on distance ranges between the features to generate the pharmacophore vector.

For example, in a case where a certain pharmacophore exists in a drug, the number of the pharmacophore is on the position of the pharmacophore vector corresponding to the pharmacophore. Alternatively, “1” indicates that the pharmacophore corresponding to the vector position of the pharmacophore vector exists in the drug, and “0” indicates that the pharmacophore corresponding to the vector position of the pharmacophore vector does not exist in the drug.

The side effect vector is used to characterize the side effect information of the drug. For example, in a case where a corresponding position in the side effect vector is “1”, it means that the drug has the side effect corresponding to the position; in a case where the corresponding position in the side effect vector is “0”, it means that the drug does not have the side effect corresponding to the position.

Targeted gene vector is used to characterize the target information that the drug can act on. The target information may be identification information specified by the gene ontology. For example, the target that Drug 1 can act on is Target 1, and the target information of Target 1 is GO:0005739.

In step 902, the prediction device determines the drug attribute information according to the drug attribute vector.

The drug attribute information includes at least one of a drug structure similarity, a pharmacophore similarity, a side effect similarity, and a GO pathway-based similarity of drugs.

The drug structure similarity refers to a degree of similarity between the drugs in chemical structure, the pharmacophore similarity refers to a degree of similarity between the drugs in pharmacophore composition, the side effect similarity refers to a degree of similarity between side effects that exist in the drugs, and the GO pathway-based similarity refers to a degree of similarity between targets that the drugs can act on.

Based on the above technical solution, the prediction device may obtain the drug attribute vector and determine the drug attribute information of the drugs in different dimensions according to the drug attribute vector, so that the prediction device subsequently determines the first drug association matrix according to the drug attribute information, thereby more comprehensively characterizing the feature information of the drugs.

The steps 901 and 902 will be described below with respect to a first drug and a second drug in the plurality of drugs.

The first drug and the second drug are drugs in the plurality of drugs. The first drug and the second drug may be the same drug, or the first drug and the second drug may be different drugs.

The process of determining the drug structure similarity between the first drug and the second drug by the prediction device is as follows.

The prediction device may obtain a drug structure vector of the first drug and a drug structure vector of the second drug, and determine the drug structure similarity between the first drug and the second drug according to the drug structure vector of the first drug and the drug structure vector of the second drug.

For example, the prediction device may obtain the drug structure vectors of drugs from the PubChem database. The prediction device may determine the drug structure similarity between the first drug and the second drug through Jaccard index or cosine similarity.

For example, the drug structure similarity satisfies the following formula 4 or formula 5.

$${S}_{i j}^{r} = \frac{\vert {x}_{i} ⋂ {x}_{j} \vert}{\vert {x}_{i} ⋃ {x}_{j} \vert} \qquad Formula 4$$

$${S}_{i j}^{r}$$

represents the arug structure similarity between the first drug and the second drug; xᵢ represents the drug structure vector of the first drug; xⱼ represents the drug structure vector of the second drug; |xᵢ∩xⱼ| represents the number of corresponding positions that are all “1” in the drug structure vectors of the first drug and the second drug (that is, the number of structures of the same type that the first drug and the second drug have); and |xᵢ∪xⱼ| represents the number of corresponding positions where “1” is present in the drug structure vectors of the first drug and the second drug (that is, the total number of structures of types that the first drun and the second drun have).

$${S}_{i j}^{r} = \frac{{x}_{i} \cdot {x}_{j}}{‖ {x}_{i} ‖ ‖ {x}_{j} ‖} \qquad Formula 5$$

$${S}_{i j}^{r}$$

represents the drug structure vector of the first drug and the second drug; xᵢ represents the drug structure vector of the second drug; xⱼ represents the drug structure vector of the second drug; xᵢ·xⱼ respresents the inner product between the drug structure vector of the first drug and the drug structure vector of the second drug; ∥xᵢ∥ represents the modulus length of the drug structure vector of the first drug; and ∥xⱼ∥ represents the modulus length of the drug structure vector of the second drug.

The process of determining the pharmacophore similarity between the first drug and the second drug by the prediction device is as follows.

The prediction device may obtain a pharmacophore vector of the first drug and a pharmacophore vector of the second drug, and determine the pharmacophore similarity between the first drug and the second drug according to the pharmacophore vector of the first drug and the pharmacophore vector of the second drug.

For example, the prediction device may determine the pharmacophore similarity between the first drug and the second drug through Jaccard index or cosine similarity. For the implementation, reference may be made to the relevant description of determining the drug structure similarity described above, which will not be described in detail here.

The process of determining the side effect similarity between the first drug and the second drug by the prediction device is as follows.

The prediction device may obtain a side effect vector of the first drug and a side effect vector of the second drug, and determine the side effect similarity between the first drug and the second drug according to the side effect vector of the first drug and the side effect vector of the second drug.

For example, the prediction device may determine the side effect similarity between the first drug and the second drug through Jaccard index or cosine similarity. For the implementation, reference may be made to the relevant description of determining the drug structure similarity described above, which will not be described in detail here.

The process of determining the GO pathway-based similarity between the first drug and the second drug by the prediction device will be described below.

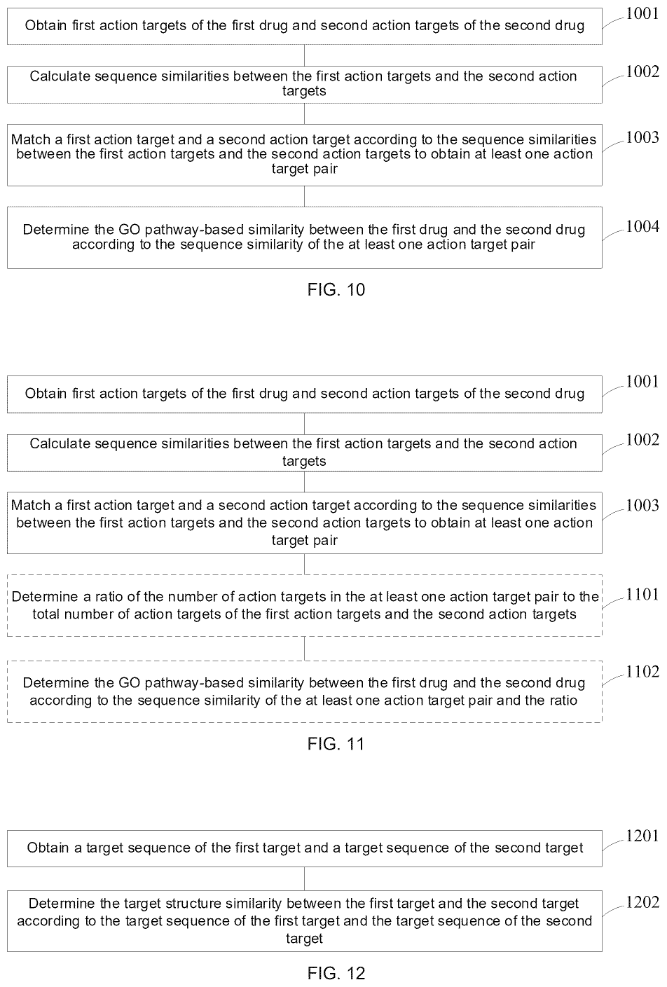

As a possible embodiment of the present disclosure, as shown in FIG. 10, before the prediction device determines the first drug association matrix, the method further includes the following steps 1001 to 1004.

In step 1001, the prediction device obtains first action targets of the first drug and second action targets of the second drug.

For example, the prediction device can obtain action targets of drugs from the GO database. The first action targets include Target A, Target B and Target C. The second action targets include Target D and Target E. The first action targets and the second action targets may be expressed in the form of targeted gene vectors. For the relevant content, reference may be made to the step 901, which will not be described in detail here.

In step 1002, the prediction device calculates sequence similarities between the first action targets and the second action targets.

In a possible implementation, the prediction device can calculate the sequence similarity between the first action target and the second action target through the GO similarity algorithm.

For example, the GO similarity algorithm is the similarity algorithm in the GOSemSim toolkit in R language.

In combination with the above example, the prediction device calculates the similarity between Target A and Target D, the similarity between Target A and Target E, the similarity between Target B and Target D, the similarity between Target B and Target E, the similarity between Target C and Target D, and the similarity between Target C and Target E.

In step 1003, the prediction device matches a first action target and a second action target according to the sequence similarities between the first action targets and the second action targets to obtain at least one action target pair.

In a possible implementation, the prediction device can determine a matching relationship between the first action target and the second action target through a matching algorithm.

For example, the matching algorithm is the Hungarian algorithm or Kuhn-Munkres algorithm (KM algorithm).

In combination with the above example, the prediction device determines that the action target pair(s) include: Target A-Target E, and Target B-Target D. Target C has no matching target.

In step 1004, the prediction device determines the GO pathway-based similarity between the first drug and the second drug according to the sequence similarity of the at least one action target pair.

For example, the GO pathway-based similarity satisfies the following formula 6.

$${S}_{G} = \frac{\sum {S}_{m a t c h}}{{N}_{m a t c h}} \qquad Formula 6$$

S_{G} represents the GO pathway-based similarity between the first drug and the second drug, S_{match} represents the sequence similarity of the two targets in the action target pair, and N_{match} represents the number of action target pairs.

Based on the above technical solution, the prediction device may use the average of similarities of the action target pairs of the first drug and the second drug as the GO pathway-based similarity between the first drug and the second drug, thereby reflecting the degree of the similarity between the first drug and the second drug in action target.

The process of determining the GO pathway-based similarity by the prediction device will be described below in combination with the step 1004.

As a possible embodiment of the present disclosure, as shown in FIGS. 10 and 11, the step 1004 may also be implemented through the following steps 1101 and 1102.

In step 1101, the prediction device determines a ratio of the number of action targets in the at least one action target pair to the total number of action targets of the first action targets and the second action targets.

In combination with the above example, the number of action targets in the action target pairs is 4, the total number of action targets of the first action targets and the second action targets is 5, and the ratio is 0.8.

In step 1102, the prediction device determines the GO pathway-based similarity between the first drug and the second drug according to the sequence similarity of the at least one action target pair and the ratio.

For example, the GO pathway-based similarity satisfies the following formula 7.

$${S}_{G} = \frac{\sum {S}_{m a t c h}}{{N}_{m a t c h}} * {N}_{G} \qquad Formula 7$$

S_{G} represents the GO pathway-based similarity between the first drug and the second drug, S_{match} represents the sequence similarity of the two targets in the action target pair, N_{match} represents the number of action target pairs, and N_{G} represents the ratio.

Based on the above technical solution, the prediction device may further consider the influence of the ratio of the number of matching targets on the GO pathway-based similarity between the first drug and the second drug when calculating the GO pathway-based similarity between the first drug and the second drug.

The process of determining the target structure similarity by the prediction device will be described below.

As a possible embodiment of the present disclosure, as shown in FIG. 12, before the prediction device determines the first target association matrix, the method further includes the following steps 1201 and 1202.

In step 1201, the prediction device obtains a target sequence of the first target and a target sequence of the second target.

The first target and the second target are targets in the plurality of targets. The first target and the second target may be the same target in the plurality of targets, or may be different targets in the plurality of targets.

The target sequence is used to characterize a chemical structure of the target. The target sequence may include at least one of primary structure, secondary structure, tertiary structure and quaternary structure.

The primary structure is taken as an example, proteins contain 20 types of amino acids, each type of amino acids is represented by a letter. For example, the target sequence of the first target is “MSFIKTFSGKHFY”, and the target sequence of the second target is “MSIKTFHGKQFY”.

In step 1202, the prediction device determines the target structure similarity between the first target and the second target according to the target sequence of the first target and the target sequence of the second target.

In a possible implementation, the prediction device can determine the target structure similarity between the first target and the second target through the edit distance (also known as the Levenshtein distance).

The edit distance refers to the minimum number of editing operations required to convert the target sequence of the first target into the target sequence of the second target. The editing operation includes the following: replacing one character with another; inserting a character; and deleting a character. Therefore, the smaller the edit distance, the greater the target structure similarity between the first target and the second target. On the contrary, the larger the edit distance, the smaller the target structure similarity between the first target and the second target.

For example, the calculation method of the edit distance is the Jaro algorithm. The target structure similarity between the first target and the second target satisfies the following formula 8.

$${d}_{J} = \{ \begin{matrix}0 & if m = 0 \\ \frac{1}{3} ( \frac{K}{\vert {s}_{1} \vert} + \frac{K}{\vert {s}_{2} \vert} + \frac{K - t}{K} ) & otherwise\end{matrix} \qquad Formula 8$$

d_{J} is the target structure similarity between the first target and the second target, |s₁| represents the number of characters in the target sequence of the first target, |s₂| represents the number of characters in the target sequence of the second target, K represents the number of matching characters in the target sequence of the first target and the target sequence of the second target, and t represents the number of transpositions required in the matching characters.

Based on the above technical solution, the prediction device may obtain the target sequence of the first target and the target sequence of the second target, and determine the target structure similarity between the first target and the second target according to the target sequence of the first target and the target sequence of the second target. In this way, the prediction device may determine the target structure similarity between pairs in the plurality of targets, so as to subsequently determine the first target association matrix.

In the embodiments of the present disclosure, the device for predicting the drug-target interaction may be divided into functional modules or functional units according to the foregoing method examples. For example, the device for predicting the drug-target interaction may be divided in a way that each functional module or unit corresponds to a function, or that two or more functions are integrated into one functional module. The integrated module may be implemented in the form of hardware or software functional module or unit. The division of modules or units in the embodiments of the present disclosure is schematic, which is merely a logical function division, and there may be other division manners in actual implementation.

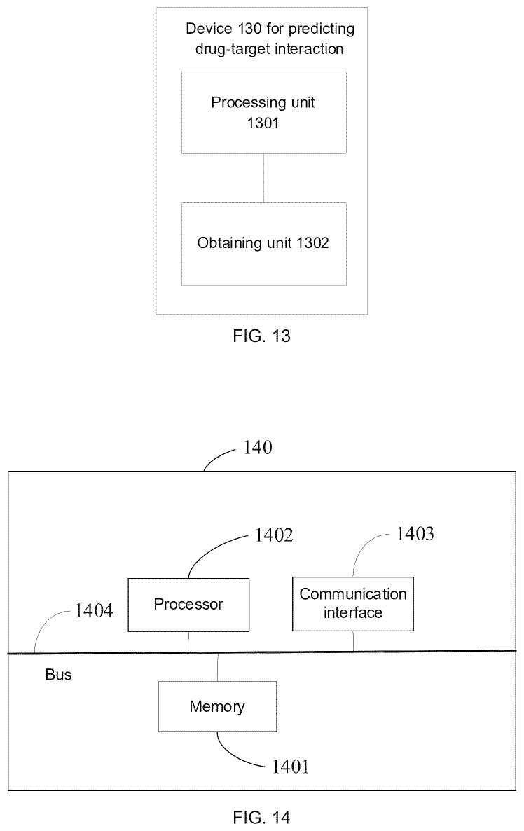

As shown in FIG. 13, which is a structural schematic diagram of a device 130 for predicting drug-target interaction, in accordance with some embodiments, the device 130 for predicting drug-target interaction includes the following.

A processing unit 1301 is configured to determine a first drug association matrix according to drug attribute information. The drug attribute information includes at least one of a drug structure similarity, a pharmacophore similarity, a side effect similarity, and a GO pathway-based similarity of drugs. The first drug association matrix is used to characterize feature information of each drug on at least one drug attribute.

The processing unit 1301 is further configured to determine a first target association matrix according to target attribute information. The target attribute information includes at least one of a target structure similarity of targets and a target interaction relationship of the targets. The first target association matrix is used to characterize feature information of each target on at least one target attribute.

The processing unit 1301 is further configured to predict the probability of interaction between the drug and the target according to the first drug association matrix and the first target association matrix.

In some embodiments, the processing unit 1301 is configured to input the drug attribute information and a drug identification vector into a first graph convolution model to obtain an initial drug association matrix. The initial drug association matrix is used to characterize feature information of the drugs on each drug attribute. The processing unit 1301 is further configured to input the initial drug association matrix into a second graph convolution model to obtain the first drug association matrix. The second graph convolution model is used to adjust the feature information of the drug according to degrees of influence of drug attributes on the drug.

In some embodiments, an obtaining unit 1302 is configured to obtain a drug attribute vector. The drug attribute vector includes at least one of drug structure vectors, pharmacophore vectors, side effect vectors and targeted gene vectors of the drugs. The processing unit 1301 is configured to determine the drug attribute information according to the drug attribute vector.

In some embodiments, the obtaining unit 1302 is configured to obtain a drug structure vector of a first drug and a drug structure vector of a second drug, the first drug and the second drug being drugs in the plurality of drugs. The processing unit 1301 is configured to determine the drug structure similarity between the first drug and the second drug according to the drug structure vector of the first drug and the drug structure vector of the second drug.

In some embodiments, the obtaining unit 1302 is configured to obtain a pharmacophore vector of a first drug and a pharmacophore vector of a second drug, the first drug and the second drug being drugs in the plurality of drugs. The processing unit 1301 is configured to determine the pharmacophore similarity between the first drug and the second drug according to the pharmacophore vector of the first drug and the pharmacophore vector of the second drug.

In some embodiments, the obtaining unit 1302 is configured to obtain a side effect vector of a first drug and a side effect vector of a second drug, the first drug and the second drug being drugs in the plurality of drugs. The processing unit 1301 is configured to determine the side effect similarity between the first drug and the second drug according to the side effect vector of the first drug and the side effect vector of the second drug.

In some embodiments, the obtaining unit 1302 is configured to obtain first action targets of a first drug and second action targets of a second drug, the first drug and the second drug being drugs in the plurality of drugs. The processing unit 1301 is configured to calculate sequence similarities between the first action targets and the second action targets. The processing unit 1301 is further configured to match a first action target and a second action target according to the sequence similarities between the first action targets and the second action targets to obtain at least one action target pair. The processing unit 1301 is further configured to determine the GO pathway-based similarity between the first drug and the second drug according to the sequence similarity of the at least one action target pair.

In some embodiments, the processing unit 1301 is configured to determine a ratio of the number of action targets in the at least one action target pair to the total number of action targets of the first action targets and the second action targets. The processing unit 1301 is further configured to determine the GO pathway-based similarity between the first drug and the second drug according to the sequence similarity of the at least one action target pair and the ratio.

In some embodiments, the processing unit 1301 is configured to input the target attribute information and a target identification vector into a third graph convolution model to obtain an initial target association matrix. The initial target association matrix is used to characterize feature information of a plurality of targets on each target attribute. The processing unit 1301 is further configured to input the initial target association matrix into a fourth graph convolution model to obtain the first target association matrix. The fourth graph convolution model is used to adjust the feature information of each target according to degrees of influence of the target attributes on each target.

In some embodiments, the obtaining unit 1302 is configured to obtain a target sequence of a first target and a target sequence of a second target, the first target and the second target being targets in the plurality of targets. The processing unit 1301 is configured to determine the target structure similarity between the first target and the second target according to the target sequence of the first target and the target sequence of the second target.

In some embodiments, the processing unit 1301 is configured to input the first drug association matrix and the first target association matrix into a first fusion model to obtain the probability of the interaction between the drug and the target.

In some embodiments, the processing unit 1301 is configured to determine a second drug association matrix according to the maximum feature value of each drug on at least one drug attribute in the first drug association matrix, the first drug association matrix including feature values of each drug on at least one drug attribute. The processing unit 1301 is further configured to determine a second target association matrix according to the maximum feature value of each target on at least one target attribute in the first target association matrix, the first target association matrix including feature values of each target on at least one target attribute. The processing unit 1301 is further configured to input the second drug association matrix and the second target association matrix into a second fusion model to obtain the probability of the interaction between the drug and the target.

When implemented by hardware, in the embodiments of the present disclosure, the obtaining unit 1302 may be integrated on a communication interface, and the processing unit 1301 may be integrated on a processor. The specific implementation is shown in FIG. 14.

FIG. 14 shows a possible structural schematic diagram of another device for predicting drug-target interaction. The device 140 for predicting drug-target interaction includes a processor 1402 and a communication interface 1403. The processor 1402 is configured to control and manage actions of the device 140 for predicting drug-target interaction (for example, perform the steps executed by the processing unit 1301 described above), and/or is configured to perform other processes of the technology described herein. The communication interface 1403 is configured to support communication between the device 140 for predicting drug-target interaction and other network entities, for example, perform the steps executed by the obtaining unit 1302 described above. The device 140 for predicting drug-target interaction may further include a memory 1401 and a bus 1404. The memory 1401 is configured to store program codes and data of the device 140 for predicting drug-target interaction.

The memory 1401 may be a memory in the device 140 for predicting drug-target interaction. The memory may include a volatile memory such as a random access memory. The memory may also include a non-volatile memory such as a read-only memory, flash memory, hard disk or solid-state disk. The memory may also include a combination of the above types of memories

The processor 1402 may be a logical block, module and circuit that implements or executes various examples described in combination with the content of the present disclosure. The processor may be a central processing unit, a general-purpose processor, a digital signal processor, an application specific integrated circuit, a field programmable gate array or any other programmable logic device, a transistor logic device, a hardware component, or any combination thereof. It may implement or execute various illustrative logical blocks, modules and circuits described in content of the present disclosure. The processor may also be a combination that implements computing functions, for example, a combination including one or more microprocessors, a combination of a digital signal processor (DSP) and a microprocessor, or the like.

The bus 1404 may be an extended industry standard architecture (EISA) bus. The bus 1404 may be divided into an address bus, a data bus, a control bus, etc. For ease of representation, only one thick line is used in FIG. 14, but it does not mean that there is only one bus or one type of bus.

The device 140 for predicting drug-target interaction in FIG. 14 may also be a chip. The chip includes one or more (including two) processors 1402 and communication interface(s) 1403.

In some embodiments, the chip further includes the memory 1401, which may include the read-only memory and the random access memory, and provide operating instructions and data to the processor 1402. Part of the memory 1401 may further include a non-volatile random access memory (NVRAM).

In some embodiments, the memory 1401 stores the following elements: execution modules, or data structures, or subsets thereof, or extended sets thereof.

In the embodiments of the present disclosure, a corresponding operation is performed by calling operating instructions stored in the memory 1401 (the operating instructions may be stored in the operating system).

From description of the above embodiments, those skilled in the art will clearly understand that, for convenience and brevity of description, an example is only given according to the above division of functional modules. In actual applications, the above functions are allocated to different functional modules as needed. That is, an internal structure of the device is divided into different functional modules to perform all or part of the functions described above. For the specific working processes of the systems, devices and units described above, reference may be made to the corresponding processes in the foregoing method embodiments, which will not be described in detail here.

Some embodiments of the present disclosure provide a computer-readable storage medium (for example, a non-transitory computer-readable storage medium), the computer-readable storage medium has stored computer program instructions, and the computer program instructions, when run on a computer (for example, the device for predicting drug-target interaction), cause the computer to perform the method for predicting drug-target interaction described in any of the above embodiments.

For example, the computer-readable storage medium may include, but is not limited to, a magnetic storage device (e.g., a hard disk, a floppy disk or a magnetic tape), an optical disk (e.g., a compact disk (CD), or a digital versatile disk (DVD)), a smart card and a flash memory device (e.g., an erasable programmable read-only memory (EPROM), a card, a stick or a key driver). Various computer-readable storage medium described in the embodiments of the present disclosure may represent one or more devices and/or other machine-readable storage medium for storing information. The term “machine-readable storage medium” may include, but is not limited to, wireless channels and various other medium capable of storing, containing and/or carrying instructions and/or data.

Some embodiments of the present disclosure further provide a computer program product, which is stored on, for example, a non-transitory computer-readable storage medium. The computer program product includes computer program instructions that, when executed on a computer (for example, the device for predicting drug-target interaction), cause the computer to perform the method for predicting drug-target interaction described in the above embodiments.

Some embodiments of the present disclosure further provide a computer program. When the computer program is executed on a computer (for example, the device for predicting drug-target interaction), the computer program causes the computer to perform the method for predicting drug-target interaction described in the above embodiments.

Some embodiments of the present disclosure further provide a device for predicting drug-target interaction, and the device includes a processor and a memory. The memory is used to store computer programs or instructions, and the processor is used to run the computer programs or instructions to implement the method for predicting drug-target interaction described in any of the above embodiments.

Beneficial effects of the computer-readable storage medium, the computer program product, the computer program, and the device for predicting drug-target interaction are same as the beneficial effects of the method for predicting drug-target interaction as described in some of the above embodiments, and details will not be repeated here.

In several embodiments provided in the present disclosure, it will be understood that the disclosed systems, devices and methods may be implemented through other manners. For example, the embodiments of the devices described above are merely exemplary. For example, the division of the units is only a logical functional division. In actual implementation, there are other division manners. For example, a plurality of units or components may be combined or integrated into another system, or some features may be ignored or not executed. On the other hand, the mutual coupling or direct coupling or communication connection shown or discussed may be an indirect coupling or communication connection through some interfaces, devices or units, and may be in an electrical, mechanical or other form.

The units described as separate components may or may not be physically separated, and the component(s) shown as units may be or may not be physical unit(s) (that is, they may be located in one place, or may be distributed to multiple network units). Some or all of the units may be selected according to actual needs to achieve the purposes of the solutions in the embodiments.

In addition, the functional units in the embodiments of the present disclosure may be integrated into one processing module or may be separate physical units, or two or more units may be integrated into one unit.

The above are only specific embodiments of the present disclosure, but the scope of protection of the present disclosure is not limited, and any person skilled in the art may conceive of variations or replacements within the technical scope of the present disclosure, which shall be included in the protection scope of the present disclosure. Therefore, the protection scope of the present disclosure should be determined by the protection scope of the claims.

What is claimed is:

1. A method for predicting drug-target interaction, comprising:
determining a first drug association matrix according to drug attribute information, wherein the drug attribute information includes at least one of a drug structure similarity, a pharmacophore similarity, a side effect similarity, and a gene ontology (GO) pathway-based similarity of a plurality of drugs; and the first drug association matrix is used to characterize feature information of each drug on at least one drug attribute;
determining a first target association matrix according to target attribute information, wherein the target attribute information includes at least one of a target structure similarity and a target interaction relationship of a plurality of targets, and the first target association matrix is used to characterize feature information of each target on at least one target attribute; and
predicting a probability of interaction between a drug and a target according to the first drug association matrix and the first target association matrix.

2. The method according to claim 1, wherein determining the first drug association matrix according to the drug attribute information, includes:
inputting the drug attribute information and a drug identification vector into a first graph convolution model to obtain an initial drug association matrix, the initial drug association matrix being used to characterize feature information of the plurality of drugs on each drug attribute; and
inputting the initial drug association matrix into a second graph convolution model to obtain the first drug association matrix, the second graph convolution model being used to adjust feature information of a drug according to degrees of influence of drug attributes on the drug.

3. The method according to claim 1, further comprising:
obtaining a drug attribute vector, the drug attribute vector including at least one of drug structure vectors, pharmacophore vectors, side effect vectors and targeted gene vectors of the plurality of drugs; and
determining the drug attribute information according to the drug attribute vector.

4. The method according to claim 1, further comprising:
obtaining a drug structure vector of a first drug and a drug structure vector of a second drug, the first drug and the second drug being drugs in the plurality of drugs; and
determining a drug structure similarity between the first drug and the second drug according to the drug structure vector of the first drug and the drug structure vector of the second drug.

5. The method according to claim 1, further comprising:
obtaining a pharmacophore vector of a first drug and a pharmacophore vector of a second drug, the first drug and the second drug being drugs in the plurality of drugs; and
determining a pharmacophore similarity between the first drug and the second drug according to the pharmacophore vector of the first drug and the pharmacophore vector of the second drug.

6. The method according to claim 1, further comprising:
obtaining a side effect vector of a first drug and a side effect vector of a second drug, the first drug and the second drug being drugs in the plurality of drugs; and
determining a side effect similarity between the first drug and the second drug according to the side effect vector of the first drug and the side effect vector of the second drug.

7. The method according to claim 1, further comprising:
obtaining first action targets of a first drug and second action targets of a second drug, the first drug and the second drug being drugs in the plurality of drugs;
calculating sequence similarities between the first action targets and the second action targets;
matching a first action target and a second action target according to the sequence similarities between the first action targets and the second action targets to obtain at least one action target pair; and
determining a GO pathway-based similarity between the first drug and the second drug according to a sequence similarity of the at least one action target pair.

8. The method according to claim 7, wherein determining the GO pathway-based similarity between the first drug and the second drug according to the sequence similarity of the at least one action target pair, includes:
determine a ratio of a number of action targets in the at least one action target pair to a total number of action targets of the first action targets and the second action targets; and
determining the GO pathway-based similarity between the first drug and the second drug according to the sequence similarity of the at least one action target pair and the ratio.

9. The method according to claim 1, wherein determining the first target association matrix according to the target attribute information, includes:
inputting the target attribute information and a target identification vector into a third graph convolution model to obtain an initial target association matrix, the initial target association matrix being used to characterize feature information of the plurality of targets on each target attribute; and
inputting the initial target association matrix into a fourth graph convolution model to obtain the first target association matrix, the fourth graph convolution model being used to adjust feature information of each target according to degrees of influence of target attributes on each target.

10. The method according to claim 1, further comprising:
obtaining a target sequence of a first target and a target sequence of a second target, the first target and the second target being targets in the plurality of targets; and
determining a target structure similarity between the first target and the second target according to the target sequence of the first target and the target sequence of the second target.

11. The method according to claim 1, wherein predicting the probability of interaction between the drug and the target according to the first drug association matrix and the first target association matrix, includes:
inputting the first drug association matrix and the first target association matrix into a first fusion model to obtain the probability of interaction between the drug and the target.

12. The method according to claim 1, wherein predicting the probability of interaction between the drug and the target according to the first drug association matrix and the first target association matrix, includes:
determining a second drug association matrix according to a maximum feature value of each drug on at least one drug attribute in the first drug association matrix, the first drug association matrix including feature values of each drug on the at least one drug attribute;
determining a second target association matrix according to a maximum feature value of each target on at least one target attribute in the first target association matrix, the first target association matrix including feature values of each target on the at least one target attribute; and
inputting the second drug association matrix and the second target association matrix into a second fusion model to obtain the probability of interaction between the drug and the target.

13. (canceled)

14. A device for predicting drug-target interaction, comprising a processor and a memory, wherein the memory is used to store computer programs or instructions, and the processor is used to run the computer programs or instructions to implement the method for predicting drug-target interaction according to claim 1.

15. A non-transitory computer-readable storage medium, wherein the computer-readable storage medium has stored instructions that, when executed on a computer, cause the computer to perform:
determining a first drug association matrix according to drug attribute information, wherein the drug attribute information includes at least one of a drug structure similarity, a pharmacophore similarity, a side effect similarity, and a gene ontology (GO) pathway-based similarity of a plurality of drugs; and the first drug association matrix is used to characterize feature information of each drug on at least one drug attribute;
determining a first target association matrix according to target attribute information, wherein the target attribute information includes at least one of a target structure similarity and a target interaction relationship of a plurality of targets, and the first target association matrix is used to characterize feature information of each target on at least one target attribute; and
predicting a probability of interaction between a drug and a target according to the first drug association matrix and the first target association matrix.

16. A computer program product, comprising computer program instructions, wherein the computer program instructions, when executed on a computer, cause the computer to perform the method for predicting drug-target interaction according to claim 1.

17. The non-transitory computer-readable storage medium according to claim 15, wherein determining the first drug association matrix according to the drug attribute information, includes:
inputting the drug attribute information and a drug identification vector into a first graph convolution model to obtain an initial drug association matrix, the initial drug association matrix being used to characterize feature information of the plurality of drugs on each drug attribute; and
inputting the initial drug association matrix into a second graph convolution model to obtain the first drug association matrix, the second graph convolution model being used to adjust feature information of a drug according to degrees of influence of drug attributes on the drug.

18. The non-transitory computer-readable storage medium according to claim 15, wherein the instructions cause the computer to further perform:
obtaining a drug attribute vector, the drug attribute vector including at least one of drug structure vectors, pharmacophore vectors, side effect vectors and targeted gene vectors of the plurality of drugs; and
determining the drug attribute information according to the drug attribute vector.

19. The non-transitory computer-readable storage medium according to claim 15, wherein determining the first target association matrix according to the target attribute information, includes:
inputting the target attribute information and a target identification vector into a third graph convolution model to obtain an initial target association matrix, the initial target association matrix being used to characterize feature information of the plurality of targets on each target attribute; and
inputting the initial target association matrix into a fourth graph convolution model to obtain the first target association matrix, the fourth graph convolution model being used to adjust feature information of each target according to degrees of influence of target attributes on each target.

20. The non-transitory computer-readable storage medium according to claim 15, wherein predicting the probability of interaction between the drug and the target according to the first drug association matrix and the first target association matrix, includes:
inputting the first drug association matrix and the first target association matrix into a first fusion model to obtain the probability of interaction between the drug and the target.

21. The non-transitory computer-readable storage medium according to claim 15, wherein predicting the probability of interaction between the drug and the target according to the first drug association matrix and the first target association matrix, includes:
determining a second drug association matrix according to a maximum feature value of each drug on at least one drug attribute in the first drug association matrix, the first drug association matrix including feature values of each drug on the at least one drug attribute;
determining a second target association matrix according to a maximum feature value of each target on at least one target attribute in the first target association matrix, the first target association matrix including feature values of each target on the at least one target attribute; and
inputting the second drug association matrix and the second target association matrix into a second fusion model to obtain the probability of interaction between the drug and the target.
